(19) United States
(12) Patent Application Publication
(10) Pub. No.: US 2020/0222227 A1
(43) Pub. Date: Jul. 16, 2020

(54) DEVICE FOR PREVENTING SLEEP APNEA AND SNORING

(71) Applicant: Yoon Seob SHIM, Incheon (KR)

(72) Inventor: Yoon Seob SHIM, Incheon (KR)

(21) Appl. No.: 16/639,038

(22) PCT Filed: Dec. 27, 2017
(86) PCT No.: PCT/KR2017/015515

§ 371 Date: Feb. 13, 2020

(30) Foreign Application Priority Data
Aug. 14, 2017 (KR) 10-2017-0103031

(51) Int. Cl. A61F 5/56

(52) U.S. Cl. CPC A61F 5/566

(57) ABSTRACT

A snoring prevention device prevents apnea symptoms and the like caused by snor-ing when a patient with severe snoring problem is sleeping, by using a dental guard having an upper jaw guard and a lower jaw guard so as to correct the upper jaw and lower jaw positions of the patient in order to prevent snoring during sleeping and specifically, the upper jaw guard and the lower jaw guard having a good comfortable fit, and an orthodontic state in which the upper jaw and the lower jaw of a patient from being artificially closed is avoided without applying an excessive force to a jaw joint such that sleep apnea and snoring can be prevented while enabling natural opening of the mouth.

### BACKGROUND

The present invention provides a snoring prevention device which can be applied to a patient suffering from severe snoring causing sleep disorders. More particularly, the present invention relates to a snoring prevention device, wherein an upper jaw guard and a lower jaw guard have a comfortable fit, and an orthodontic state in which the upper jaw and the lower jaw of a patient are artificially closed is avoided without applying an excessive force to a jaw joint such that sleep apnea and snoring can be prevented while enabling natural opening of the mouth.

A jaw joint disc is located between a facial bone and a jaw bone, and the structure of a jaw joint (or a temporomandibular joint) moving according to a method of rotating relative to the jaw joint disc by opening and closing a lower jaw is moved relatively upward when human body ages or lower teeth wear out, and the jaw joint disc may come out forward, thereby leading to instability.

Such an abnormal position of the jaw joint is recognized as a factor that may cause a variety of diseases, and induces snoring and acts as a factor that causes sleep disorder or sleep apnea.

Commonly defined snoring may be called a noise phenomenon during respiration occurring by vibrating a structure around the soft palate (a relatively soft back part in a mouth ceiling) relaxed while respiratory airflow during sleep passes through an airway narrowed by various causes and the uvula (Adam's apple, which has a drooping shape below the center of the soft palate of the mouth and is also referred to as Hyeon-ongsu).

That is, as a result of juxtaposition of an upper portion of the tongue or velum and posterior pharyngeal wall during sleep, airway obstruction may occur.

In general, the diameter of the airway of an adult is known as an average of about 13 mm, and when an airway diameter of about 9 mm or less is maintained, oxygen supply is insufficient, which may lead to a state in which normal breathing is impossible.

The phenomenon of such oxygen supply insufficiency often occurs during sleep, and may lead to snoring.

The reason for snoring during sleep may vary, but one of the reasons is that breathing through the nose does occur normally. In particular, the abnormal position of the jaw joint during sleep is pointed out as a major cause for snoring.

That is, when a lower jaw falls back during sleep, falls back even in normal times, or the neck is thick, severe snoring tends to occur. This is because space around the Adam's apple is narrow and the securing of the width of an airway for breathing is difficult.

Particularly, severe snoring prevents sound sleep at night and, in some cases, may be accompanied by sleep apnea.

Such obstructive sleep apnea leads to a fatal disease in which breathing stops during sleep for potentially 10 seconds or more, or more than 300 times during sleep at night. Such a symptom causes fatigue and sleepiness during the day, and if the sleep apnea is left untreated, it may develop into complications of the cardiopulmonary system, accompanied by hypertension, arrhythmia, or sudden death. Therefore, it is desirable to diagnose and treat the sleep apnea as early as possible.

Various methods have been proposed to alleviate or prevent a snoring symptom that may cause such results. For example, snoring prevention surgery is performed or a snoring prevention pillow is used.

But in the case of surgery, various devices designed to provide positive ventilation methods and lower jaw advancement, and the advancement of the tongue, or the combination thereof are used.

Particularly, one of the most common surgical methods is uvulopalatoplasty, which is surgery to remove a portion of the velum so as to prevent the closure of the throat airway during sleep.

However, such a surgical method requires several-day hospitalization and a several-week recovery cycle, and thus is applied to patients most difficult to be cured. Further, the surgical method may be accompanied by unexpected side effects, so such an irreversible treatment is not preferred for general patients.

In the meanwhile, various other surgical methods have been proposed, including bone fixture insertion or the implantation of a magnet in the Adam's apple, but are still not preferred for patients.

Meanwhile, an example unlike the various surgical methods is positive pressure breathing, the most common of which is continuous positive airway pressure (CPAP), which is the method of supplying pressurized air to a sleeping patient by using a mask.

The use of such a CPAP may have an immediate effect on the quality of sleep and life in general, but it may be seen as one of the methods that general patients avoid due to the configuration of masks and hoses accompanying the CPAP device.

Accordingly, these surgical or treatment methods proposed are greatly avoided in consideration of effective aspects and preferences according to a patient's condition, and as for the orthodontic pillow providing a relatively simple configuration described above, the pillow supporting a rear portion of the neck may be removed away from the body and produce no effect when a patient's body tosses and turns during sleep.

To complement the above-described surgical or treatment methods, there is treatment or correction in which a jaw joint is targeted.

There is a correction device designed to achieve the lower jaw advancement through the correction of the jaw joint, and relying on the lower jaw advancement has been known to significantly reduce the tendency of an upper portion of the tongue to relax against the posterior pharyngeal wall, thereby relieving snoring and sleep apnea in many cases.

For example, as a preferred method, a lower jaw advancement supports the tongue forward, and in order to relieve the above-described symptoms, the lower jaw advancement of about 5 to 10 mm is required.

The devices for such a lower jaw advancement or orthodontic tools are basically configured to include a pair of bow-shaped tooth coupling units, which are independent of each other, that is, a dental guard.

Each of the tooth coupling units has a generally flat surface in contact with the complementary surface of each other, is designed to be precisely engaged with all or a portion of the tooth arrangement of the upper jaw or the lower jaw if appropriate, and has the engaging surface of an opposing tooth arrangement.

Among the preceding patents, “snoring helper” is disclosed in Korean Utility Model Application Publication No. 20-2010-0006504, and according to the prior art described above, after the snoring helper including a C-shaped body having a predetermined elastic force is inserted into the nose, the snoring helper is prevented from being removed from the nose, and further, functions to help enlarging the area of the nostrils, thereby preventing snoring.

However, this method has a problem in that it may interfere with getting a sufficient sleep by causing a breathing disorder during sleep.

Unlike this, as a means for preventing snoring by being inserted into the structure of the mouth, “Snore and sleep apnea preventing device” is disclosed in Korean Patent No. 10-0596129, and the device is inserted into the mouth to prevent snoring without disturbing breathing.

However, in the prior art, a seating groove of teeth is formed in advance, and thus does not match the mouth structure of various users. Accordingly, the device is removed from the mouth structure or is not securely fixed thereto during sleep, so the prior art has limitation in achieving desired effects.

Each of the prior arts described above has problems as described above and can be applicable only to snoring prevention. Accordingly, when there is a problem with the jaw joint, the device of the prior art cannot perform the correction of the jaw joint although the device functions to prevent snoring, or when the above-mentioned snoring prevention device worn during sleep is used for a long term, a patient having a structural problem in the jaw joint may have a problem that it may be difficult to correct the jaw joint by missing the treatment time or by the structural problem becoming chronic.

Accordingly, in view of this, that is, for the prevention of snoring, the relief of apnea symptoms, and the correction of jaw joint described above, “Snore preventing and temporomandibular joint adjusting device” is disclosed in Korean Patent Application Publication No. 10-2012-0033386.

The device disclosed in the patent includes: a rigid central frame having a through hole provided at a middle thereof and having the shape of an arc, which is the shape of teeth arrangement; an upper member provided by being bonded to an upper surface of the rigid central frame; and a lower member provided by being bonded to a lower surface of the rigid central frame, wherein the upper member and the lower member are made soft by being heated during use and can be molded to fit the shape of user's teeth by the user biting the upper member and the lower member, so the above-described problems can be solved. However, the device has inconvenience in that it is required to be newly adjusted or molded according to a correction process.

In addition, if a patient adjusts the device corresponding to a jaw joint correction state for himself or herself, accurate correction is difficult or correction may be performed slantingly. Of course, the possibility that such a wrong correction is stuck and the correcting cannot be performed may not be excluded, and further, if a patient adjusts such a device, or performs the correction arbitrarily rather than visiting a dentist, it is impossible to obtain accurate treatment data of the patient.

By supplementing the prior art of various embodiments described above, “Replaceable clip type mandibular advancement device for snoring and sleep apnea treatment” is disclosed in Korean Patent No. 10-1590813 by the applicant.

According to the above patent technology, the replaceable clip type mandibular advancement device includes a dental guard having an upper jaw guard and a lower jaw guard combined with an upper jaw and a lower jaw of a patient, respectively, a combination part having an upper jaw combination part and a lower jaw combination part provided at end parts of the upper jaw guard and the lower jaw guard of the dental guard, respectively, and the adjuster fitted to the lower jaw combination part and adjusting a position of the jaw of the patient, wherein the upper jaw combination part of an end part of the upper jaw guard has a protrusion part protruding to a side surface thereof, and a front of the protrusion part supports the adjuster at a rear thereof. Accordingly, a right adjuster can be selected and replaced at right time to increase therapeutic and corrective effects. (See registered patent publication)

Furthermore, “Self-adjustable jaw joint calibration devices for snoring, sleep apnea treatment” is disclosed in Korean Patent No. 10-1710840 by the applicant.

As for the patent, the self-adjustable jaw joint calibration device for snoring, sleep apnea treatment including a dental guard which has an upper jaw guard and a lower jaw guard combined with an upper jaw and a lower jaw of a patient, respectively, the lower jaw guard having a tooth combination part provided at an upper side of a front thereof, and upper and lower jaw guide parts guiding advancement of the lower jaw guard by combining a lower surface of the upper jaw guard with an upper surface of the lower jaw guard,

the self-adjustable jaw joint calibration device includes: a tension combination part continuing to a lower surface of the upper jaw guard from a front surface thereof and having a penetrating member having a through hole formed therethrough, an elastic member extending by being bent from a lower surface of a seating groove constituting a rear side of the penetrating member in a direction of the penetrating member, a holding protrusion provided at a lower end of an end part of the elastic member, a combination hole formed through a middle portion of the elastic member, and a fixing combination part fitted to the tension combination part and combined with the tooth combination part by tension fixing,

wherein the fixing combination part includes an extension piece extending to a rear of a press part of a front surface thereof, a tension support piece having a tension force provided by being cut to be spaced apart from the extension piece to each of the left and right of the extension piece, a first protrusion part bent to a lower end of an end part of the extension piece, and a second protrusion part protruding to an outer surface of an end part of the tension support piece, wherein

while the lower jaw guard and the upper jaw guard are combined with each other, the lower jaw guard is gradually moved forward from the upper jaw guard to be fixed such that a position of a jaw of the patient is adjusted. (See registered patent publication).

As described above, while the applicant has been continuously developing and implementing various methods and configurations for improving the condition of patients having severe snoring and an apnea symptom during sleep, the applicant recognized that the above-described prior arts may damage the jaw joint due to the characteristic of the jaw joint opening naturally during patient's sleep.

When the upper jaw and the lower jaw of the patient are artificially fixed so as not to open in a state in which the lower jaw among the jaw joints is advanced by a predetermined distance, an unexpected problem may occur.

Accordingly, the applicant recognized that a structure in which a state in which the lower jaw is moved forward by a predetermined distance from the upper jaw during sleep is maintained and the natural phenomenon that the upper jaw and the lower jaw are naturally opened by the relaxation of the jaw joint during sleep are not controlled artificially may correspond to needs of a patient.

In addition, conventional orthodontic devices are produced uniformly regardless of the structure, condition, and size of the mouth of a patient, and cannot be applied to the various structures and sizes of the mouth of each patient. Accordingly, when each of the devices is worn by a patient, the feeling of wearing is not natural, so when worn for a long time or term, the device may cause deformation of teeth and diseases accompanied by inflammation on the surface of the gum.

### SUMMARY OF THE INVENTION

The present invention has been made keeping in mind the above problems,

and is intended to provide a snoring prevention device, which is worn in the mouth of a patient suffering from severe snoring accompanied by apnea during sleep and maintains a forward-moved state of a lower jaw while maintaining the characteristic of an upper jaw and the lower jaw of the patient naturally opening at a predetermined angle during sleep such that a snoring symptom accompanied by apnea during sleep can be relieved and corrected.

In addition, the present invention is intended to provide a snoring prevention device, which can be applied irrespective of the structure of the mouth of a patient, that is, tooth arrangement in the mouth and the opening angle of a gum, thereby ensuring a comfortable fit in the mouths of all patients, preventing the possibility of inflammatory disease of gum surface tissues caused by wear side effects, and preventing tooth deformation that may be caused by wear for long time and term.

In order to accomplish the above objectives, the present invention uses a dental guard consisting of an upper jaw guard and a lower jaw guard to correct positions of an upper jaw and a lower jaw of a patient so as to prevent snoring during sleep so that the patient suffering from severe snoring is prevented from having an apnea symptom caused by the snoring during sleep.

According to the present invention, the snoring prevention device is worn in the mouth of a patient suffering from severe snoring accompanied by apnea during sleep and maintains a forward-moved state of a lower jaw while maintaining the characteristic of an upper jaw and the lower jaw of the patient naturally opening at a predetermined angle during sleep such that a snoring symptom accompanied by apnea during sleep can be relieved and corrected.

In addition, the present invention is intended to provide a snoring prevention device, which can be applied irrespective of the structure of the mouth of a patient, that is, tooth arrangement in the mouth and the opening angle of a gum, thereby ensuring a comfortable fit in the mouths of all patients, preventing the possibility of inflammatory disease of gum surface tissues caused by wear side effects, and preventing tooth deformation that may be caused by wear for long time and term.

### BRIEF DESCRIPTION OF DRAWINGS

FIGS. 1 to 3 illustrate states of an impression clip member initially applied to perform the impression work of the tooth arrangement of a patient, in which

FIG. 1 is a perspective view illustrating a state in which the impression clip member and upper and lower jaw guards are combined with each other;

FIG. 2 is an exploded perspective view illustrating a state in which the upper jaw guard and the lower jaw guard of FIG. 1 are separated from each other;

FIG. 3 is an exploded perspective view illustrating a state in which the impression clip member is separated from the upper jaw guard of FIG. 2;

FIGS. 4 to 10 illustrate configurations for the position adjustment of the upper and lower jaw guards by using a clip member after the tooth arrangement of a patient is impressed by using the impression clip member of FIGS. 1 to 3, in which

FIG. 4 is an exploded perspective view illustrating a state in which the upper jaw guard having the clip member applied thereto and the lower jaw guard are separated from each other;

FIG. 5 is a bottom exploded perspective view illustrating a state in which the lower jaw guard of FIG. 4 is separated from a lower surface covering member of the lower jaw;

FIG. 6 is an exploded perspective view illustrating a state in which the clip member is separated from the upper jaw guard of FIG. 4;

FIG. 7 is a side view roughly illustrating the upper and lower jaw guards in a state in which the upper jaw guard and the lower jaw guard of FIG. 4 are worn in the mouth of a patient;

FIG. 8 is a side view roughly illustrating a state of a lower jaw maintained despite the natural opening phenomenon of the upper and lower jaws after the upper and lower jaw guards are worn in the mouth of the patient;

FIG. 9 is a view roughly illustrating that advancement of the lower jaw can be selected by selecting a clip member having a selected length of an entire length of a removable combination part of the clip member to change a state of the advancement of the lower jaw; and

FIG. 10 is a view illustrating the provision of the clip member of the present invention having the removable combination part of various lengths as a set provided in the present invention to select the state of the advancement of the lower jaw of FIG. 9.

### DETAILED DESCRIPTION OF THE INVENTION

As a best mode for practicing the present invention,

A device for preventing sleep apnea and snoring, the snoring prevention device having a dental guard 1 composed of an upper jaw guard 10 and a lower jaw guard 20 combined with an upper jaw and a lower jaw of a patient, respectively, the device including:

cut grooves 121, 221 multiply provided by being cut vertically at equal intervals along an inner side of a front end of each of the upper jaw guard 10 and the lower jaw guard 20 and spread from side to side according to a condition of a mouth of a patient such that the device is worn in the mouth,

a protrusion part 250 provided upward at each of opposite sides of the lower jaw guard 20,

a clip fixing part 150 provided at each of side surfaces of opposite ends of the upper jaw guard 10, the clip fixing part being in contact with a rear end surface of the protrusion part 250, and

a clip member 30 roundly covering a front surface of the upper jaw guard 10 and having a removable combination part 310 provided at each of opposite ends thereof so as to be combined with and removed from the clip fixing part 150.

Throughout the drawings, the same reference numerals will refer to the same or like parts. Specific structural and functional descriptions of an exemplary embodiment of the present invention disclosed herein are only for illustrative purposes of the embodiment of the present invention. The present invention may be embodied in many different forms without departing from the spirit and significant characteristics of the present invention. Therefore, the present invention is intended to cover not only the exemplary embodiment, but also various alternatives, modifications, equivalents and other embodiments that may be included within the spirit and scope of the present invention as defined by the appended claims. Terms or words used in the specification and claims are not limited to meanings that are commonly understood or are defined in dictionaries, and should be interpreted as having meanings and concepts that are consistent with the context of the present invention, based on the principle that an inventor may properly define concepts of words so as to describe his or her invention in the best mode.

Accordingly, although the exemplary embodiment of the present invention has been described for illustrative purposes, those skilled in the art will appreciate that various modifications, additions and substitutions are possible without departing from the scope and spirit of the invention as disclosed in the accompanying claims.

In addition, unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Hereinbelow, the exemplary embodiment of the present invention will be described with reference to the accompanying drawings.

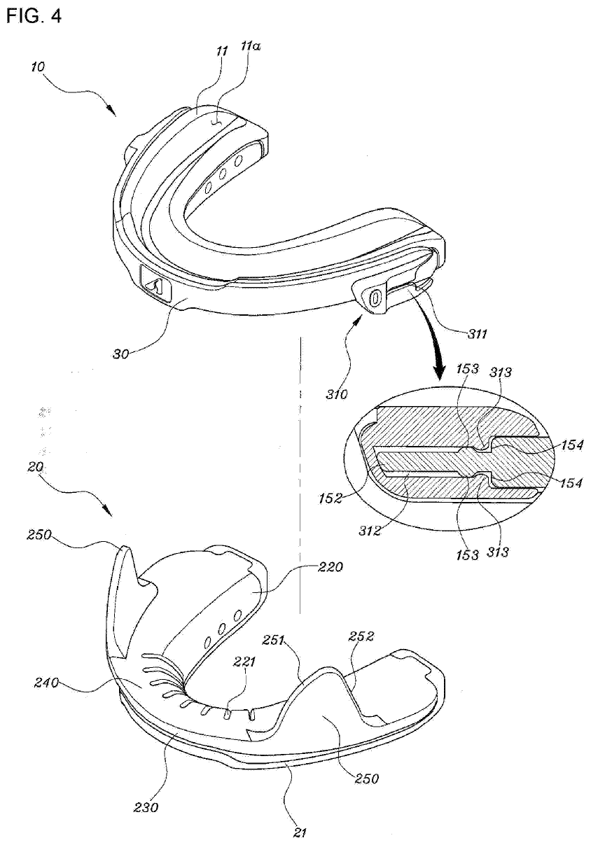

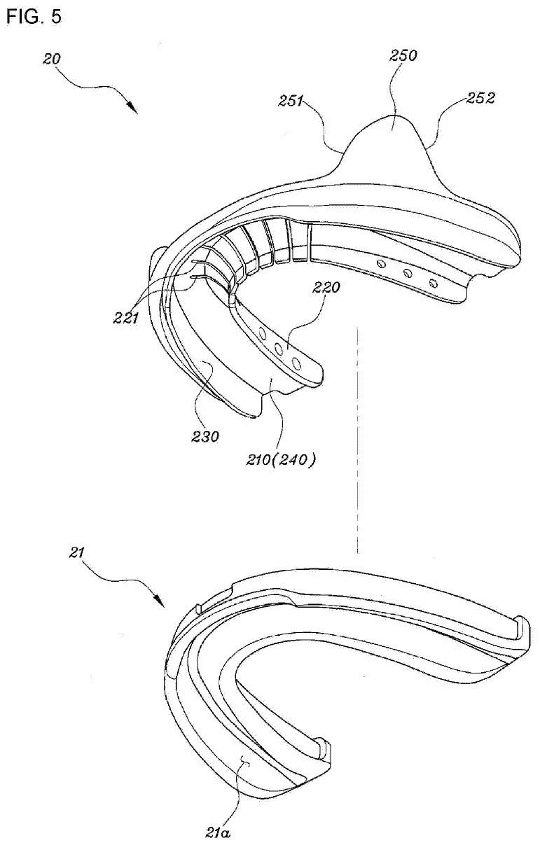

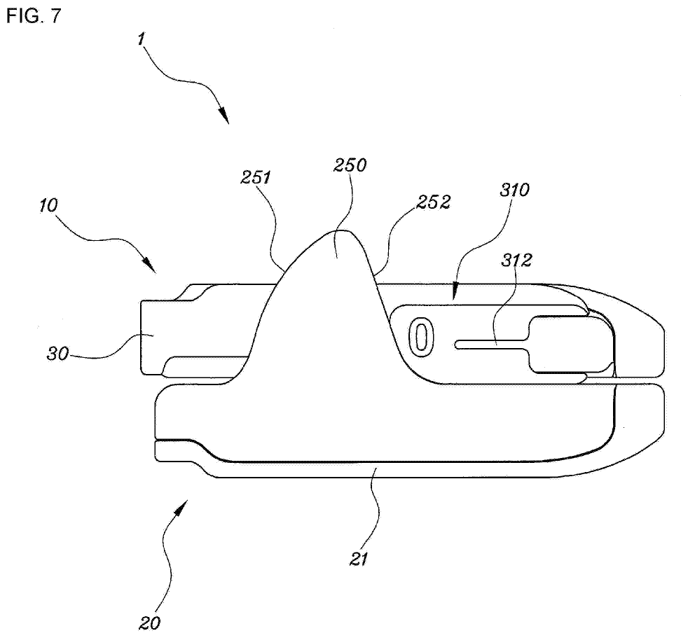

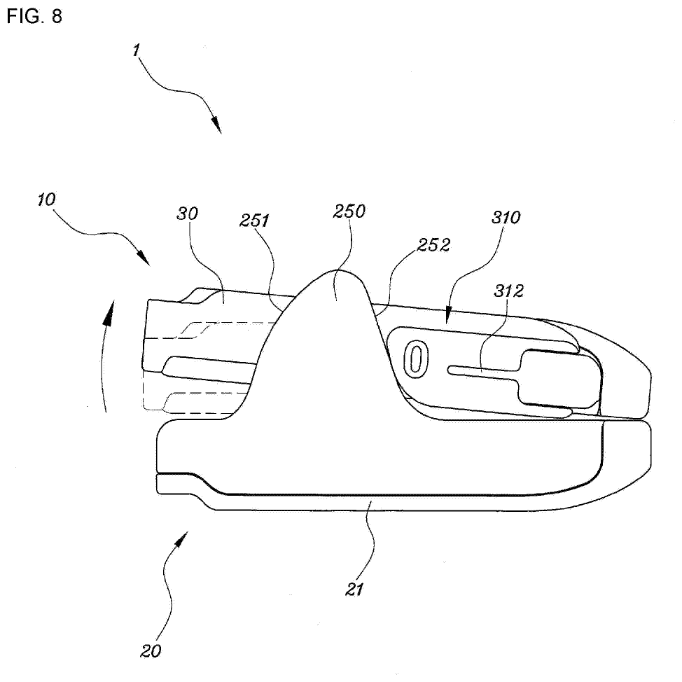

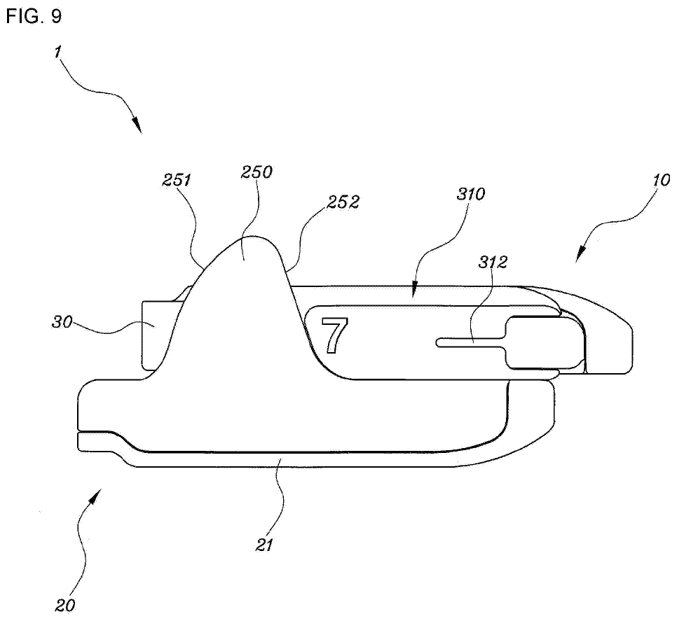

As illustrated in FIGS. 4 to 10 of the present invention, the snoring prevention device of the present invention is worn in the mouth of a patient suffering from obstructive sleep apnea having snoring accompanied by an apnea symptom during sleep, whereby the lower jaw is maintained in a state of moving more forward by a predetermined degree than the upper jaw such that a snoring symptom is suppressed during sleep.

To this end, the present invention provides the upper jaw guard 10 and the lower jaw guard 20 as a set, which are inserted into and worn in the mouth of a patient.

An upper surface covering member 11 of an upper jaw and a lower surface covering member 21 of a lower jaw, which are shaped by a shape memory material consisting of a thermoplastic resin, are combined with and fixed to an upper surface of the upper jaw guard 10 and a lower surface of the lower jaw guard 20, respectively.

An upper surface of the upper surface covering member 11 of an upper jaw and a lower surface of the lower surface covering member 21 of a lower jaw are portions being in contact with teeth of a patient, and each of tooth-fixing grooves 11a and 21a is formed to correspond to the tooth arrangement of a patient.

Configuration for the combination of the upper surface covering member 11 of an upper jaw with the upper jaw guard 10 and the combination of the lower surface covering member 21 of a lower jaw with the lower jaw guard 20 may have various embodiments.

For example, a protrusion may be provided on a lower surface of the upper surface covering member 11 of an upper jaw and a combination groove may be provided in an upper seating groove 110 of the upper jaw guard 10 to be combined with the protrusion. A protrusion may be provided on an upper surface of the lower surface covering member 21 of a lower jaw and a combination groove may be provided in a lower seating groove 210 of the lower jaw guard 20 to be combined with the protrusion.

Besides, configuration for combination between upper and lower cover members 11 and 21 and the upper and lower jaw guards 10 and 20 can be performed by various methods and embodiments.

In addition, the upper surface covering member 11 of an upper jaw and the lower surface covering member 21 of a lower jaw, which may be changed according to the state of the tooth arrangement of a patient, are manufactured after imitating the tooth arrangement of a patient and then can be combined with and fixed to each other to be used by the configuration according to the embodiment described above.

That is, the upper jaw guard 10 and the lower jaw guard 20 of the present invention can be supplied in mass production, and the upper surface covering member 11 of an upper jaw and the lower surface covering member 21 of a lower jaw can be formed and supplied by using the shape memory material consisting of a thermoplastic resin according to the tooth arrangement of a patient who visits a hospital, so burden on the patient can be greatly eliminated.

Forming the upper surface covering member 11 of an upper jaw and the lower surface covering member 21 of a lower jaw by using the shape memory material as described above is to allow the snoring prevention device to be restored to an initial shape when a predetermined time lapses after a sleep state stops and the snoring prevention device of the present invention is removed from the mouth although the shape of the device may be changed due to temperature change when the snoring prevention device of the present invention is worn in the mouth of a patient.

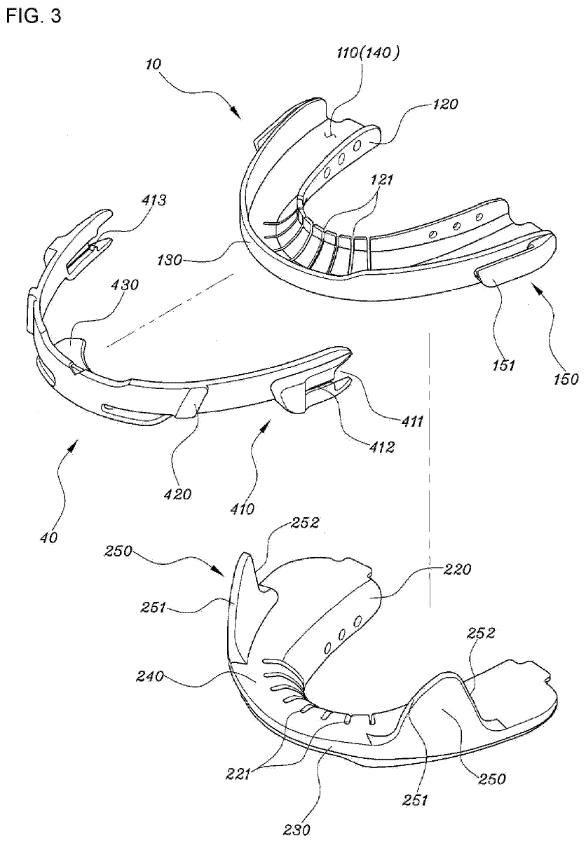

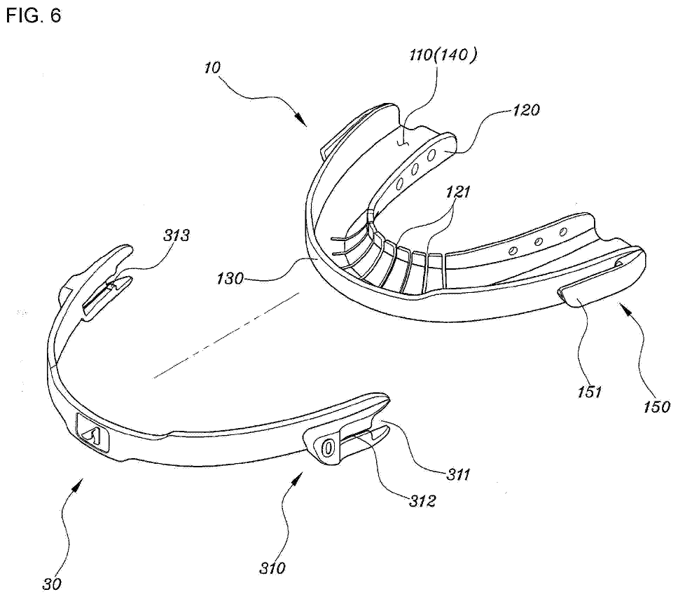

Such an upper jaw guard 10 has a predetermined shape as illustrated in FIGS. 3 and 6, and the seating groove 110 is formed upward such that the upper surface covering member 11 of an upper jaw is fitted over and combined with the seating groove, and the upper jaw guard 10 is cut at the same intervals along an inner side of a front end thereof, so the upper jaw guard 10 may be spread or retracted to be worn on the patient's mouth according to the condition of patient's mouth.

That is, when describing the upper jaw guard 10 of the present invention by dividing the upper jaw guard 10 into an inner part 120, an outer part 130, and a bottom part 140 relative to the seating groove 110 for convenience, the cut grooves 121 are provided to be spaced apart at the equal intervals from each other so as to continue to the bottom part 140 from the inner part 120 constituting the inner side of the front end of the upper jaw guard 10.

Meanwhile, the upper jaw guard 10 includes the clip fixing part 150 provided at a side surface of each of opposite ends thereof, the clip fixing part being in contact with the rear end surface (the rear end surface when viewed in drawings) of the protrusion part 250 of the lower jaw guard 20.

The clip fixing part 150 is a means for fixing the clip member 30 and functions to maintain a forward-moved state of the lower jaw guard 20.

As illustrated in FIG. 3, the clip fixing part 150 is configured to include an insertion guide 151 provided by protruding from each of side surfaces of opposite side ends of the upper jaw guard 10, a first end of the insertion guide having a closed shape and a second end thereof being open, such that an end part of the clip member 30 is guided to be fitted into the insertion guide, wherein a horizontal support bar 152 is provided by continuing horizontally from an inner surface of the insertion guide 151 to the side surface of the upper jaw guard 10; a protruding combination part 153 is provided on each of upper and lower surfaces of the horizontal support bar 152; and a vertical step 154 is provided by continuing from the protruding combination part 153 so that the end part of the clip member 30 is fixed to and combined with the clip fixing part after being fitted thereto.

As for such a protruding combination part 153, a holding combination part 313, 413 is formed at a portion of continuing to a spaced guide groove 312, 412 from an open groove 311, 411 of each of the clip member 30 and an impression clip member 40, which are described hereinafter, and can be engaged with the protruding combination part 153 provided by protruding from each of upper and lower surfaces of the horizontal support bar 152 constituting the clip fixing part 150 of the upper jaw guard 10 and be combined with the protruding combination part 153 by the step 154.

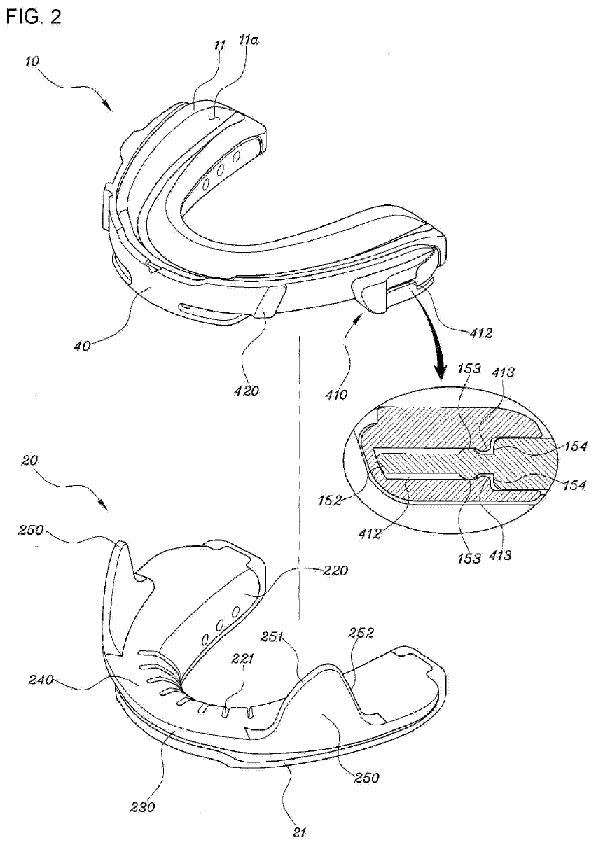

The lower jaw guard 20 has a predetermined shape as illustrated in FIGS. 2, 3, and 4, and is configured to face the upper jaw guard 10. The seating groove 210 is formed downward in the lower jaw guard such that the lower surface covering member 21 is fitted over and combined with the seating groove, and the lower jaw guard 20 is cut at the same intervals along an inner side of a front end thereof, so the lower jaw guard 20 may be spread or retracted to be worn on the patient's mouth according to the condition of patient's mouth.

That is, as the upper jaw guard 10 described above, when describing the lower jaw guard 20 of the present invention by dividing the lower jaw guard 20 into an inner part 220, an outer part 230, and a ceiling part 240 relative to the seating groove 210 for convenience, the cut grooves 221 are provided to be spaced apart at the equal intervals from each other so as to continue to the ceiling part 240 from the inner part 220 constituting the inner side of the front end of the lower jaw guard 20.

Meanwhile, as illustrated in FIG. 2, the protrusion part 250 is provided upward at each of opposite sides of the lower jaw guard 20 and has a triangular shape as a whole.

The protrusion part 250 is configured to include a gently curved surface part 251 having a gentle inclination angle at a front side thereof and a sharply curved surface part 252 having a sharp inclination angle at a rear side thereof, the inclination angle of the sharply curved surface part 252 being greater than the inclination angle of the gently curved surface part 251.

The sharply curved surface part 252 and the removable combination part 310 of the clip member 30 combined with the clip fixing part 150 of the upper jaw guard 10 described above are maintained to be in contact with each other, so a rearward movement of the lower jaw guard 20 can be controlled.

Meanwhile, as described above, the position adjustment of the upper jaw guard 10 and the lower jaw guard 20 and the maintenance of the fixed states of the corresponding positions thereof are made possible by the clip member 30.

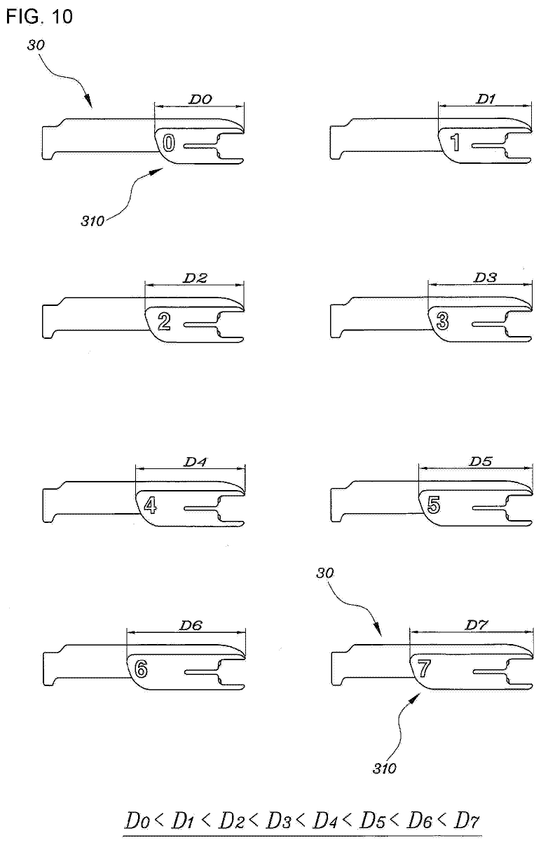

The clip member 30 is preferably configured to be selectively adopted according to the condition of a patient as illustrated in FIG. 10.

For example, the clip member 30 and the removable combination part 310 are configured to be provided as a set by allowing an entire length (D0, D1, D2˜D7) of the removable combination part 310 provided at each of opposite ends of the clip member 30 to be different, and is worn by adjusting a length of lower jaw advancement depending on a snoring condition of the patient.

First, the configuration of the clip member 30 will be described.

As described above, the clip member 30 is combined with the clip fixing part 150 provided at opposite ends of the upper jaw guard 10, and a contact state between the clip member 30 and the sharply curved surface part 252 of the rear end surface of the protrusion part 250 of the lower jaw guard 20 is maintained, so that the forward-moved state of the lower jaw guard 20 can be fixed.

Such a clip member 30 is combined with the upper jaw guard 10 as if it is surrounding the front surface of the upper jaw guard 10, has the shape of an arc in the entire configuration thereof as illustrated in FIG. 6, has elasticity, and includes the removable combination part 310 provided at a side surface of each of the opposite ends of the clip member as described above.

To fit the removable combination part 310 over the clip fixing part 150 provided at an outer surface of each of the opposite ends of the upper jaw guard 10 described above, an end portion of a front side of the spaced guide groove 312 is configured to be closed after the spaced guide groove 312, which is narrow, is formed by continuing from the open groove 311 open at an end portion of a rear side of the removable combination part 310.

In addition, a portion having a closed shape, which is an end portion of a front side of the removable combination part 310, is configured in a curved shape and is in contact with the sharply curved surface part 252 of the protrusion part 250 of the lower jaw guard 20, so that while the clip member 30 is combined with the upper jaw guard 10 by the removable combination part 310, the rearward movement of the lower jaw guard 20 is controlled.

In addition, as described above, multiple clip members 30 are provided by differently setting the entire length of the removable combination part 310, and although clip members 30 different from each other in the entire length of the removable combination part 310 are provided, the cut length of the spaced guide groove 312 is required to be formed to be the same.

That is, the cut length of the spaced guide groove 312 is required to be formed to be the same as the length of the horizontal support bar 152 corresponding thereto provided at the clip fixing part 150 of the upper jaw guard 10. The length of the advancement of the lower jaw guard 20 can be selected and fixed due to the variability of the entire length of the removable combination part, and the fixed state of such an advancement length can be selected according to the condition of a patient suffering from snoring.

Meanwhile, to use the device for preventing sleep apnea and snoring of the present invention using the configurations described above, an impression process of imitating the tooth arrangement of a patient is required to obtain the upper surface covering member 11 of an upper jaw and the lower surface covering member 21 of a lower jaw, which correspond to the tooth arrangement of the patient.

In order to proceed with such an impression process, an impression work is required to be performed with upper and lower jaws of a patient maintained at normal states.

That is, a clip member for determining an initial set position of each of the upper jaw guard 10 and the lower jaw guard 20 is required to perform the impression work, and in this case, the clip member is different in characteristic from the clip member 30 worn during sleep to relieve the snoring symptom.

Accordingly, in the present invention, the clip member for such an impression work will be described by being separately named as the impression clip member 40.

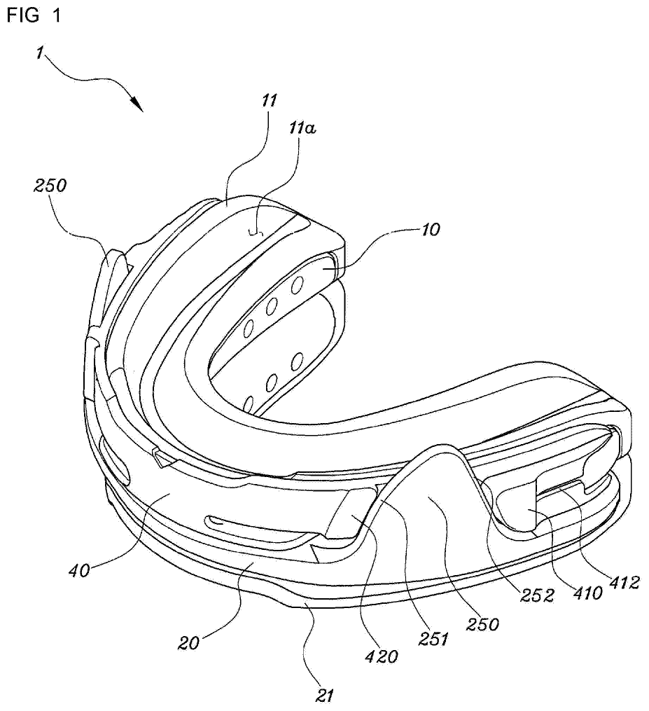

As described above, the impression clip member 40 for obtaining the impression of patient's teeth in a normal state has the same configuration as the configuration of the clip member 30 described above. However, as illustrated in FIGS. 1 to 3, the upper jaw guard 10 and the lower jaw guard 20 are required to maintain fixed states, in which the forward and rearward movements are not performed.

To this end, the impression clip member 40 roundly covers the front surface of the upper jaw guard 10 and has a removable combination part 410 such that each of opposite ends of the impression clip member 40 is combined with and removed from the clip fixing part 150. The basic configuration of the impression clip member 40 is similar to the configuration of the clip member 30 described above.

In addition, the open groove 411 is also provided to be open at an end portion of a rear side of the removable combination part 410 of the impression clip member 40, and after the spaced guide groove 412, which is narrow, is provided to communicate with the open groove 411 by continuing from the open groove 411, an end portion of a front side of the spaced guide groove 412 is configured to be closed.

In this case, a portion having a closed shape, which is an end portion of a front side of the removable combination part 410, is configured in a curved shape and is in contact with the sharply curved surface part 252 of the protrusion part 250 of the lower jaw guard 20 so that while the impression clip member 40 is combined with the upper jaw guard 10 by the removable combination part 410, the rearward movement of the lower jaw guard 20 is controlled.

In addition, unlike the clip member 30 described above, the impression clip member 40 includes a stopper part 420 formed by protruding from an outer surface thereof at a position spaced apart from the removable combination part 410 along an outer surface of the impression clip member 40.

A space spaced apart between the removable combination part 410 and the stopper part 420 is used as a space to fit the protrusion part 250 described above thereinto.

That is, during the impression work for imitating the state of the tooth arrangement of the mouth of a patient suffering from snoring, the protrusion part 250 formed at each of the opposite sides of the lower jaw guard 20 is fitted between the removable combination part 410 and the stopper part 420 to maintain the fixed states of the upper and lower jaws, so the impression work of a patient is facilitated.

The stopper part 420 is preferably configured in a gently curved shape to correspond the gently curved surface part 251 of the protrusion part 250 so as to be in close contact with each other.

In addition, a horizontal extension piece 430 is preferably provided by extending horizontally from an inner side of a front lower end of the impression clip member 40 to support the upper jaw guard 10 during the impression work.

After the impression work is completed by the impression clip member 40 having such a configuration in a state of an initial mouth of a patient to obtain the upper surface covering member 11 of an upper jaw and the lower surface covering member 21 of a lower jaw imitating the tooth arrangement, any one of the clip members 30 described above, that is, a clip member 30 having a selected length of the entire length of the removable combination part 310 is selected and is worn in the mouth of a patient during sleep such that the snoring condition can be relieved or corrected.

Although the present invention is described by the limited embodiment and drawings, the present invention is not limited to this and may be modified or changed by those skilled in the art within the spirit of the art of the present invention and the range of the claims below.

Accordingly, the technical spirit in the present invention should be understood by the scope of the claims described below, and it will be apparent that all equivalent or equivalent modifications belong to the scope of the technical spirit of the present invention.

The snoring prevention device of the present invention can be applied irrespective of the structure of the mouth of a patient, that is, tooth arrangement in the mouth and the opening angle of a gum, thereby ensuring a comfortable fit in the mouths of all patients, preventing the possibility of inflammatory disease of gum surface tissues caused by wear side effects, and preventing tooth deformation that may be caused by wear for long time and term. Further, the snoring prevention device has industrial applicability in that the device relieves and corrects a snoring symptom accompanied by apnea during patient's sleep by maintaining the forward-moved state of the lower jaw.

1. A device for preventing sleep apnea and snoring, the snoring prevention device having a dental guard (1) consisting of an upper jaw guard (10) and a lower jaw guard (20) combined with an upper jaw and a lower jaw of a patient, respectively, the device comprising:
cut grooves (121, 221) multiply provided by being cut vertically at equal intervals along an inner side of a front end of each of the upper jaw guard (10) and the lower jaw guard (20) and spread from side to side according to a condition of a mouth of a patient such that the device is worn in the mouth; a protrusion part (250) provided upward at each of opposite sides of the lower jaw guard (20); a clip fixing part (150) provided at each of side surfaces of opposite ends of the upper jaw guard (10), the clip fixing part being in contact with a rear end surface of the protrusion part (250); and a clip member (30) roundly covering a front surface of the upper jaw guard (10) and having a removable combination part (310) provided at each of opposite ends thereof such that the removable combination part is combined with and removed from the clip fixing part (150).

2. The device of claim 1, wherein the cut grooves (121) are provided to be spaced apart at the equal intervals from each other so as to continue to a bottom part (140) from an inner part (120) constituting the inner side of the front end of the upper jaw guard (10), and the cut grooves (221) are provided to be spaced apart at the equal intervals from each other so as to continue to a ceiling part (240) from an inner part (220) constituting the inner side of the front end of the lower jaw guard (20) corresponding to the upper jaw guard (10).

3. The device of claim 1, wherein the clip fixing part (150) comprises an insertion guide (151) provided by protruding from each of side surfaces of opposite side ends of the upper jaw guard (10), a first end of the insertion guide having a closed shape and a second end thereof being open, such that an end part of the clip member (30) is guided to be fitted into the insertion guide, wherein a horizontal support bar (152) is provided by continuing horizontally from an inner surface of the insertion guide (151) to the side surface of the upper jaw guard (10); a protruding combination part (153) is provided on each of upper and lower surfaces of the horizontal support bar (152); and a vertical step (154) is provided by continuing from the protruding combination part (153) so that the end part of the clip member (30) is fixed to and combined with the clip fixing part after being fitted thereto.

4. The device of claim 1, wherein the protrusion part (250) is configured to include a gently curved surface part (251) having a gentle inclination angle at a front side thereof and a sharply curved surface part (252) having a sharp inclination angle at a rear side thereof, the inclination angle of the sharply curved surface part (252) being greater than the inclination angle of the gently curved surface part (251).

5. The device of claim 1, wherein an end portion of a front side of a spaced guide groove (312) is configured to be closed after the spaced guide groove (312), which is narrow, is provided to communicate with an open groove (311) by continuing from the open groove (311) open at an end portion of a rear side of the removable combination part (310) of the clip member (30).

6. The device of claim 5, wherein the clip member (30) and the removable combination part (310) are configured to be provided as a set by allowing an entire length (D0, D1, D2˜D7) of the removable combination part (310) provided at each of opposite ends of the clip member (30) to be different so as to be worn by adjusting a length of lower jaw advancement according to a snoring condition of the patient.

7. The device of claim 1, wherein in an impression process of imitating tooth arrangement of the patient to obtain an upper surface covering member (11) of the upper jaw and a lower surface covering member (21) of the lower jaw, an impression clip member (40) is provided to determine initial set positions of the upper jaw guard (10) and the lower jaw guard (20), the impression clip member (40) roundly covering the front surface of the upper jaw guard (10) and having a removable combination part (410) provided at each of opposite ends thereof such that the removable combination part is combined with and removed from the clip fixing part (150), an open groove (411) is provided to be open at an end portion of a rear side of the removable combination part (410) of the impression clip member (40), and after a spaced guide groove (412), which is narrow, is provided to communicate with the open groove (411) by continuing from the open groove (411), an end portion of a front side of the spaced guide groove (412) is configured to be closed, a portion having a closed shape, which is an end portion of a front side of the removable combination part (410), is configured in a curved shape and is in contact with a sharply curved surface part (252) of the protrusion part (250) of the lower jaw guard (20) so that while the impression clip member (40) is combined with the upper jaw guard (10) by the removable combination part (410), a rearward movement of the lower jaw guard (20) is controlled, and a stopper part (420) is provided by protruding from an outer surface of the impression clip member (40) at a position spaced apart from the removable combination part (410) along the outer surface of the impression clip member (40) so that the protrusion part (250) is fixed by being fitted between the removable combination part (410) and the stopper part (420).

8. The device of claim 7, wherein the stopper part (420) is configured in a gently curved shape to correspond a gently curved surface part (251) of the protrusion part (250) so as to be in close contact with each other.

9. The device of claim 7, wherein a horizontal extension piece (430) is provided by extending horizontally from an inner side of a front lower end of the impression clip member (40) to support the upper jaw guard (10) during an impression work.

10. The device of claim 5, wherein a holding combination part (313) is provided at a portion of continuing to the spaced guide groove (312) from the open groove (311), and is engaged with and combined with a protruding combination part (153) provided by protruding from each of upper and lower surfaces of a horizontal support bar (152) constituting the clip fixing part (150) of the upper jaw guard (10).

11. The device of claim 7, wherein a holding combination part (313) is provided at a portion of continuing to the spaced guide groove (312) from the open groove (311), and is engaged with and combined with a protruding combination part (153) provided by protruding from each of upper and lower surfaces of a horizontal support bar (152) constituting the clip fixing part (150) of the upper jaw guard (10).
